(19) United States
(12) Patent Application Publication
(10) Pub. No.: US 2026/0257911 A1
(43) Pub. Date: Sep. 3, 2026

(54) COMPOSITION FOR LIQUID ORGANIC HYDROGEN CARRIER COMPRISING MIXTURE OF MONOBENZYLTOLUENE ISOMERS, AND METHOD FOR STORING AND RELEASING HYDROGEN BY USING SAME

(71) Applicant: IUCF-HYU (INDUSTRY-UNIVERSITY COOPERATION FOUNDATION HANYANG UNIVERSITY), Seoul (KR)

(72) Inventors: Young Woong SUH, Seoul (KR); Tae Wan KIM, Incheon (KR); Yeong In JO, Hwaseong-si (KR)

(73) Assignee: IUCF-HYU (INDUSTRY-UNIVERSITY COOPERATION FOUNDATION HANYANG UNIVERSITY), Seoul (KR)

(21) Appl. No.: 18/846,769

(22) PCT Filed: Mar. 8, 2023
(86) PCT No.: PCT/KR2023/003143

§ 371 Date: Sep. 13, 2024

(30) Foreign Application Priority Data
Mar. 17, 2022 (KR) 10-2022-0033252

(51) Int. Cl. C01B 3/0015; C07C 5/10; C07C 5/32; C07C 15/16

(52) U.S. Cl. CPC C01B 3/0015; C07C 5/10; C07C 5/325; C07C 15/16

(57) ABSTRACT

The present disclosure describes a technology that uses a mixture of monobenzyltoluene isomers of controlled composition as a liquid organic hydrogen carrier on the basis of the properties of individual structural isomers (ortho-, meta-, and para-isomers) of monobenzyltoluene, and is capable of storing and releasing hydrogen with a high selective conversion rate and high efficiency by suppressing factors that inhibit a stable hydrogenation/dehydrogenation cycle.

### TECHNICAL FIELD

The present disclosure relates to a composition for a liquid organic hydrogen carrier containing a monobenzyltoluene isomer mixture and a method of storing and releasing hydrogen using the same. More particularly, the present disclosure relates to storage and release of hydrogen with high selective conversion and high efficiency by suppressing factors that impede stable hydrogenation/dehydrogenation cycles, with the use of a monobenzyltoluene isomer mixture of controlled composition as a liquid organic hydrogen carrier based on the properties of individual regioisomers (ortho, meta, and para isomers) of monobenzyltoluene.

### BACKGROUND ART

As environmental pollution problems, such as decreased reserves of fossil fuels, global warming caused by carbon dioxide and pollutants generated during combustion, and the like, are becoming more serious, global interest in and demand for development of alternative energy is explosively increasing.

As alternative energy sources, renewable energy such as wind power, tidal power, geothermal energy, hydrogen energy, solar energy, etc. is in the spotlight, and each of the energy sources listed above has advantages and disadvantages. In this regard, efficient demand and supply of renewable energy are difficult due to instability of energy sources depending on time and the natural environment, so in order to effectively replace conventional fossil fuels, there is a need to develop technologies for storing and supplying surplus energy such that energy may be stably supplied.

From this perspective, hydrogen energy is receiving particular attention because it (i) has the highest energy content per unit mass, (ii) generates only water and no other harmful byproducts during combustion, (iii) is advantageous in terms of reuse because water, the main source of hydrogen, is abundant in nature and conversion into water after use is possible, and (iv) is suitable for attaining competitiveness in the precision industries and IT industries with distributed energy resources that use hydrogen as fuel. Moreover, hydrogen may be produced using other renewable energy such as solar energy, wind power, etc. and may be widely applied not only to various energy sources but also to other industrial fields (e.g., various petrochemical fields), and is emerging as a key energy source of the future at home and abroad.

In general, research and development of hydrogen energy is largely divided into production, delivery (transport) and storage of hydrogen. Hydrogen has high energy storage capacity per mass but low energy storage capacity per volume, so developing technologies for efficiently storing hydrogen are critical to the practical use of hydrogen energy. For example, materials having a mass storage density of 10 wt % or more at 77 K are known, but the volume storage density of these materials is 40 g/L, which is low for use as an actual hydrogen storage material. Therefore, thorough research is ongoing into technologies for storing as much hydrogen as possible in a storage medium that is as light and compact as possible.

In this regard, many countries have invested a lot of money in research and development over the years, and efficient hydrogen storage methods that have been researched and developed to date are the CGH2 method, which compresses hydrogen at a pressure of 700 bar, and the LH2 method, which stores liquid hydrogen by liquefaction at −253° C. However, this pressurization or liquefaction method must maintain a low temperature, and the density of liquid hydrogen is also low at 71.2 kg/m³.

Recently, hydrogen storage technologies have been developed by reversible catalytic hydrogenation/dehydrogenation using organic materials, for example liquid organic hydrogen carriers (LOHCs) (U.S. Pat. No. 7,901,491, etc.). LOHC technology has advantages of being able not only to store 5 wt % or more of hydrogen, which is recommended by the U.S. Department of Energy (DOE), but also to operate in a low pressure range, easily regenerate stored materials, and the like. Moreover, it is possible to provide a practical hydrogen storage system that enables efficient transport and storage using existing liquid fuel transport and storage infrastructure due to advantages of liquid fossil fuels such as ease of handling and high energy density (Energy Rev., 6 (2002) 141, Energy Environ. Sci., 8 (2015) 1035-1045).

There are known technologies using pi-conjugated substrate compounds as LOHC materials (e.g., U.S. Pat. No. 7,351,395, etc.), but it is difficult to obtain consistent reactivity due to pi-conjugation, and such compounds are mostly present as solids and are thus used in a mixed form. In this regard, representative examples of LOHC materials include benzyltoluene, dibenzyltoluene, etc. (e.g., Korean Patent Application Publication No. 2015-97558, etc.), and monobenzyltoluene is of particular interest.

In this regard, U.S. Patent Application Publication No. 2015/0266731 discloses the use of, as an LOHC, a monobenzyltoluene isomer mixture, commercially available heat transfer oil under the trade name Marlotherm LH (ortho:meta:para=46:5:49). This product is advantageous in view of economic efficiency, such as being easily available, etc. compared to pure materials in specific isomer forms.

However, when the monobenzyltoluene isomer mixture is used as an LOHC, limitations are imposed on achieving rapid hydrogen storage/release efficiency. In particular, as hydrogen storage/release cycles progress, hydrogenation/dehydrogenation efficiency decreases, which is undesirable.

Therefore, a method capable of performing stable hydrogenation/dehydrogenation by identifying hydrogenation/dehydrogenation tendency of monobenzyltoluene and suppressing a phenomenon that lowers hydrogen storage/release cycle efficiency is required.

### DISCLOSURE

### Technical Problem

An embodiment of the present disclosure provides a method capable of achieving good hydrogen storage/release efficiency over a long period of time based on identified hydrogenation/dehydrogenation tendency of each isomer of monobenzyltoluene as a liquid organic hydrogen carrier.

Another embodiment of the present disclosure provides a liquid organic hydrogen carrier suitable for reversible hydrogen storage/release through hydrogenation/dehydrogenation.

### Technical Solution

A first aspect of the present disclosure provides a process of reversibly storing and releasing hydrogen, comprising:
a) providing a monobenzyltoluene (MBT) isomer mixture having a para-isomer content of at least 50 mol %;
b) subjecting the monobenzyltoluene (MBT) isomer mixture to hydrogenation to store hydrogen; and
c) subjecting the hydrogenated monobenzyltoluene (MBT) isomer mixture to dehydrogenation to release hydrogen therefrom,
in which hydrogen is stored or hydrogen is released by alternately performing hydrogenation and dehydrogenation, and

a methylfluorene content in the dehydrogenated monobenzyltoluene (MBT) isomer mixture is maintained at less than 10 mol %.

In an embodiment, a halogen content in the monobenzyltoluene (MBT) isomer mixture may be maintained at less than 30 ppm (w/w).

In an embodiment, the monobenzyltoluene (MBT) isomer mixture may comprise 42 mol % or less of ortho-monobenzyltoluene (o-MBT), 15 mol % or less of meta-monobenzyltoluene (m-MBT), and 55 to 90 mol % of para-monobenzyltoluene (p-MBT).

In an embodiment, step b) may be carried out in the presence of a catalyst, and the catalyst may comprise a support comprising a porous inorganic oxide, and at least one active metal selected from the group consisting of nickel, cobalt, palladium, platinum, iridium, ruthenium, rhodium, copper, gold, rhenium, zinc, molybdenum, tin, manganese, tungsten, and germanium.

In an embodiment, a content of the active metal in the catalyst used in step b) may be set in the range of 0.1 to 50 wt %.

In an embodiment, the porous inorganic oxide in the catalyst used in step b) may be at least one selected from the group consisting of alumina, silica, silica-alumina, aluminum phosphate, zirconia, titania, ceria, and yttria.

In an embodiment, step c) may be carried out in the presence of a catalyst, and the catalyst may comprise a support including a porous inorganic oxide, an active metal, and sulfur.

In an embodiment, the content of the active metal in the catalyst used in step c) may be determined in the range of 0.1 to 10 wt % and a sulfur content may be determined in the range of 0.001 to 2 wt %.

In an embodiment, the molar ratio of sulfur/active metal in the catalyst used in step c) may be determined in the range of 0.1 to 1.

In an embodiment, the porous inorganic oxide in the catalyst used in step c) may be alumina and the active metal may be platinum.

A second aspect of the present disclosure provides a liquid organic hydrogen carrier composition for reversible hydrogen storage/release, comprising:
42 mol % or less of ortho-monobenzyltoluene (o-MBT);
15 mol % or less of meta-monobenzyltoluene (m-MBT); and
55 to 90 mol % of para-monobenzyltoluene (p-MBT),
in which a methylfluorene content is controlled to be less than 10 mol % and a halogen content is controlled to be less than 30 ppm (w/w).

In an embodiment, the halogen may be chlorine.

### Advantageous Effects

According to an embodiment of the present disclosure, a monobenzyltoluene isomer mixture of controlled composition based on the effect or tendency of monobenzyltoluene (MBT) regioisomers such as ortho, meta, and para isomers on reversible hydrogenation (hydrogen storage) and dehydrogenation (hydrogen release) is used as an LOHC, and furthermore, a decrease in hydrogen storage/release efficiency that occurs during hydrogenation/dehydrogenation cycles can be suppressed. Additionally, the use of a catalyst that suppresses the formation of methylfluorene during dehydrogenation provides the advantage of further improving hydrogen storage/release reversibility over a long period of time.

### DESCRIPTION OF DRAWINGS

FIG. 1 schematically shows synthesis of ortho-, meta-, and para-monobenzyltoluene by reacting 2-, 3-, and 4-methylbenzyl chloride with excess benzene in the presence of a homogeneous catalyst;

FIG. 2 is a graph showing results of GC analysis of three isomers (ortho, meta, and para) of monobenzyltoluene in hydrogen-rich (H₁₂-MBT) and hydrogen-lean (H₀-MBT) states, and an isomer mixture corresponding to commercially available Marlotherm LH;

FIGS. 3a to 3c are graphs showing results of ¹H NMR (left) and ¹³C NMR (right) analysis of the three synthesized monobenzyltoluene isomers;

FIGS. 4a to 4c are graphs showing results of hydrogenation of the three synthesized monobenzyltoluene isomers depending on the concentration of Cl (reaction conditions: 10 g of reactant, hydrogen pressure of 50 bar, M/R of 0.15 mol %, 150° C., and 1200 rpm);

FIGS. 5a to 5c are graphs showing results of dehydrogenation of the three synthesized monobenzyltoluene isomers depending on the concentration of Cl (reaction conditions: 1.5 g of reactant, 1 bar, M/R of 0.60 mol %, 260° C., 215 min, and 200 rpm);

FIGS. 6a to 6C are graphs showing results of hydrogenation performed after removing Cl from the three synthesized monobenzyltoluene isomers (reaction conditions: 10 g of reactant, hydrogen pressure of 50 bar, M/R of 0.035 mol %, and 1200 rpm);

FIGS. 7a to 7c are graphs showing results of dehydrogenation performed after removing Cl from the three synthesized monobenzyltoluene isomers (reaction conditions: 1.5 g of reactant, 1 bar, M/R of 0.60 mol %, 215 min, and 200 rpm) and;

FIGS. 8a and 8b show Arrhenius plots of hydrogenation and dehydrogenation performed after removing Cl from the three synthesized monobenzyltoluene isomers (respective conditions for hydrogenation and dehydrogenation are the same as in FIGS. 6 and 7);

FIGS. 9a to 9c show results of GC-FID analysis of products of dehydrogenation performed after removing Cl from the three synthesized monobenzyltoluene isomers (reaction conditions: 1.5 g of reactant, 1 bar, M/R of 2.40 mol %, 260° C., 215 min, and 200 rpm);

FIGS. 10a to 10c are graphs showing results of evaluation of adsorption energy for Pt(111), Ru(0001), and γ-Al₂O₃ (110) sites of H₀-MBT, cis-H₁₂-MBT, and trans-H₁₂-MBT based on DFT;

FIGS. 11a and 11b are graphs showing results of thermogravimetric analysis of catalysts (Al₂O₃ and Ru/Al₂O₃) adsorbed with three monobenzyltoluene isomers;

FIGS. 12a and 12b are graphs showing results of hydrogenation and dehydrogenation of a binary physical mixture of two monobenzyltoluene isomers (para and meta) (hydrogenation conditions: 10 g of reactant, hydrogen pressure of 50 bar, M/R of 0.035 mol %, 110° C., and 1200 rpm; dehydrogenation conditions: 1.5 g of reactant, 1 bar, M/R of 0.60 mol %, 240° C., 215 min, and 200 rpm);

FIGS. 13a and 13b are graphs showing results of hydrogenation and dehydrogenation of a binary physical mixture of two monobenzyltoluene isomers (para and ortho) (hydrogenation conditions: 10 g of reactant, hydrogen pressure of 50 bar, M/R of 0.035 mol %, 110° C., and 1200 rpm; dehydrogenation conditions: 1.5 g of reactant, 1 bar, M/R of 0.60 mol %, 240° C., 215 min, and 200 rpm);

FIGS. 14a and 14b are graphs showing results of hydrogenation and dehydrogenation of a ternary physical mixture of three monobenzyltoluene isomers (ortho, meta, and para) (hydrogenation conditions: 10 g of reactant, hydrogen pressure of 50 bar, M/R of 0.035 mol %, 110° C., and 1200 rpm; dehydrogenation conditions: 1.5 g of reactant, 1 bar, M/R of 0.60 mol %, 240° C., 215 min, and 200 rpm);

FIG. 15 shows results of GC analysis in the hydrogen-rich state (H₁₂-MBT) and the hydrogen-lean state (H₀-MBT) of selective isomer compositions (p72 and p55) prepared by a synthesis process of increasing the composition of para-monobenzyltoluene;

FIGS. 16a to 16d show (a, c) results of hydrogen storage and release of selective isomer compositions (p72 and p55) prepared by a synthesis process of increasing the content of para-monobenzyltoluene and MLH-sim as a comparative example, and (b, d) Arrhenius plots thereof (hydrogenation conditions: 10 g of reactant, hydrogen pressure of 50 bar, M/R of 0.035 mol %, 130° C., and 1200 rpm; dehydrogenation conditions: 1.5 g of reactant, 1 bar, M/R of 2.40 mol %, 260° C., 215 min, and 200 rpm);

FIGS. 17a and 17b are graphs showing results of GC analysis of products obtained by dehydrogenation of para-monobenzyltoluene (a: hydrogen-rich state (p-H₁₂-MBT); b: hydrogen-lean state (p-H₀-MBT));

FIG. 18 is photographs showing apparent colors of para-monobenzyltoluene and products obtained by dehydrogenation using the same as a reactant (reaction conditions: 10 g of reactant, M/R of 2.40 mol %, 270° C., 215 min, and 200 rpm);

FIG. 19 is a graph showing results of hydrogenation depending on the concentration of methylfluorene (MF) in para-monobenzyltoluene (reaction conditions: 10 g of reactant, hydrogen pressure of 50 bar, M/R of 0.035 mol %, 130° C., and 1200 rpm);

FIGS. 20a and 20b are graphs showing results of dehydrogenation of para-monobenzyltoluene in the presence of each of a Pt/Al₂O₃ catalyst and a Pt—S/Al₂O₃ catalyst and GC analysis of the obtained products (reaction conditions: 10 g of reactant, 1 bar, M/R of 2.40 mol % (Pt/Al₂O₃ catalyst) and 0.15 mol % (Pt—S/Al₂O₃ catalyst), 270° C., 215 min, and 200 rpm);

FIG. 21 is a graph showing results of stability evaluation of the selective isomer composition (p72) prepared by a synthesis process of increasing the composition of para-monobenzyltoluene (hydrogenation conditions: 10 g of reactant, hydrogen pressure of 50 bar, M/R of 0.035 mol % for 1^{st} run and 0.14 mol % for subsequent runs, 130° C., and 1200 rpm; dehydrogenation conditions: 10 g of reactant, 1 bar, M/R of 0.15 mol %, 270° C., 215 min, and 200 rpm;

FIGS. 22a and 22b are graphs showing results of hydrogen storage (hydrogenation) and hydrogen release (dehydrogenation) tests depending on the type of reactant (hydrogenation conditions: 10 g of reactant, hydrogen pressure of 50 bar, M/R of 0.035 mol %, 130° C., and 1200 rpm; dehydrogenation conditions: 1.5 g of reactant, 1 bar, M/R of 2.40 mol %, 260° C., 215 min, and 200 rpm);

FIG. 23 shows results of GC analysis of products obtained by repeating hydrogen storage/release of para-monobenzyltoluene (hydrogenation conditions: 10 g of reactant, hydrogen pressure of 50 bar, M/R of 0.035 mol % for 1^{st} run and 0.14 mol % for subsequent runs, 130° C., reaction time of 44 min for 1^{st} run and 82 min for subsequent runs, and 1200 rpm; dehydrogenation conditions: 10 g of reactant, 1 bar, M/R of 0.15 mol %, 270° C., 215 min, and 200 rpm); and

FIG. 24 shows results of GC analysis of products obtained by repeating hydrogen storage/release of the selective isomer composition (p72) prepared by a synthesis process of increasing the composition of para-monobenzyltoluene (hydrogenation conditions: 10 g of reactant, hydrogen pressure of 50 bar, M/R of 0.035 mol % for 1^{st} run and 0.14 mol % for subsequent runs, 130° C., reaction time of 43 min for 1^{st} run and 40 min for subsequent runs, and 1200 rpm; dehydrogenation conditions: 10 g of reactant, 1 bar, M/R of 0.15 mol %, 270° C., 215 min, and 200 rpm).

### BEST MODE

The present disclosure can be realized in its entirety based on the following description. It should be understood that the following description is given of preferred embodiments of the present disclosure, and the present disclosure is not necessarily limited thereto. In addition, the accompanying drawings are provided to aid understanding of the present disclosure, and the present disclosure is not limited thereto.

Terms used herein may be defined as follows.

The term “liquid organic hydrogen carrier (LOHC)” may refer to an organic fluid that may be hydrogenated in the presence of a catalyst for easy storage and transport and may then be dehydrogenated in the presence of a catalyst.

The term “hydrogenation” may refer to a reaction that increases the hydrogen content in a compound by chemically adding hydrogen to at least a portion of the compound by bringing the compound into contact with a catalyst under supply of hydrogen.

The term “dehydrogenation” may refer to a reaction in which hydrogen in a compound is removed and further the removed hydrogen is released from the compound.

The term “support” may refer to a material (for example, a solid material) with a high specific surface area to which a catalytically active component is attached.

The term “monobenzyltoluene” may refer to a compound in which a benzyl group is located in the ortho, meta, and/or para positions relative to a methyl group.

The term “monobenzyltoluene isomer mixture” may be understood to include not only a binary mixture containing a combination of at least two selected from among regioisomers (ortho, meta, and para isomers), but also a ternary mixture containing three regioisomers.

The term “hydrogen-lean monobenzyltoluene” may refer to dehydrogenated monobenzyltoluene, as represented by H₀-MBT, while the term “hydrogen-rich monobenzyltoluene” may refer to fully hydrogenated monobenzyltoluene, storing 12 hydrogen atoms, as represented by H₁₂-MBT.

Herein, when a numerical range is specified as a lower limit and/or an upper limit, it may be understood that any sub-combination within the numerical range is also disclosed. For example, when described as “1 to 5,” it may include not only 1, 2, 3, 4, and 5, but also any sub-combinations therebetween.

When any component or member is described herein as being “connected” to another component or member, it may be understood that it includes not only cases where it is directly connected to the other component or member, but also cases where it is connected by the intervention of a further component or member, unless stated otherwise.

Similarly, the term “contact” may be understood to include not only direct contact, but also contact by the intervention of a further component or member.

When any component is “included,” it means that another component may be further included, unless stated otherwise.

In the present disclosure, the compositional ratio is controlled by identifying the tendency of each of three regioisomers (ortho, meta, and para isomers) of monobenzyltoluene (MBT), one of LOHCs, shown in reversible or repetitive hydrogenation/dehydrogenation, thereby improving reaction efficiency and also suppressing a decrease in efficiency of the overall hydrogen storage/release process due to impurities and/or irreversible byproducts, ultimately achieving stable hydrogen storage/release efficiency.

### Liquid Organic Hydrogen Carrier (Hydrogen Storage Material)

The present inventors have found that the rates of hydrogenation (hydrogen storage) and dehydrogenation (hydrogen release) are distinct for each of the three regioisomers of monobenzyltoluene. In an LOHC, which is used as a means for hydrogen storage, high hydrogen storage/release efficiency is the most important factor, and a monobenzyltoluene isomer mixture of controlled composition based on results of evaluation of hydrogen storage/release activity of individual isomers is provided, thereby improving hydrogenation/dehydrogenation efficiency.

For the monobenzyltoluene isomer mixture, commercially available heat transfer oil such as Marlotherm LH is conventionally used, but the use of the commercial product makes it difficult to prepare an isomer mixture in which the ratio between multiple isomers is controlled, and a method of separating and purifying specific isomers is also difficult to implement physicochemically.

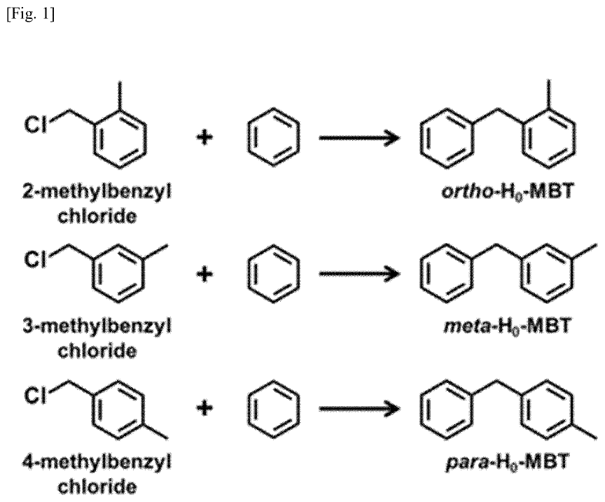

In this regard, in an embodiment, in order to prepare a monobenzyltoluene isomer mixture of controlled composition, an ortho, meta, or para-monobenzyltoluene isomer may be obtained by reacting a compound having an alkyl group attached to a specific position of a benzene ring in a methylbenzyl halide compound represented by General Formula 1 below (i.e., 2-methylbenzyl halide, 3-methylbenzyl halide, or 4-methylbenzyl halide) with benzene (alkylation; FIG. 1).

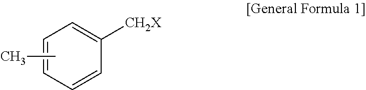

Here, CH₃ may be located in the ortho, meta, or para position relative to CH₂X, and X may be fluorine, chlorine, bromine, or iodine, for example chlorine or bromine, for example chlorine (Cl).

In the reaction described above, benzene reacting with methylbenzyl halide may be used in excess, and the amount of benzene used may be adjusted in the range of, for example, about 1 to 25 mol, for example about 2 to 20 mol, for example 3 to 15 mol, for example about 5 to 10 mol, relative to methylbenzyl halide.

According to an embodiment, the reaction may be performed in the presence of a homogeneous catalyst, and such a catalyst may be at least one selected from among Fe(III) halides, such as FeCl₃, FeBr₃, etc. For example, FeCl₃ may be used. The amount of catalyst used is not particularly limited, but may be set in the range of, for example, about 0.001 to 0.8 mol, for example about 0.005 to 0.5 mol, for example about 0.01 to 0.2 mol, relative to methylbenzyl halide.

Optionally, the reaction may be carried out using a solvent, which may be selected from among types that are not reactive with reactants and products. For example, the solvent may be at least one selected from the group consisting of aliphatic hydrocarbons (e.g., aliphatic hydrocarbons having 6 to 9 carbon atoms), halogenated hydrocarbons (e.g., methylene chloride, tetrachloroethane, etc.), and halogenated aromatic hydrocarbons (e.g., dichlorobenzene, trichlorobenzene, etc.).

In addition, the reaction may be carried out at a temperature of, for example, about 5 to 120° C., for example about 10 to 100° C., for example about 20 to 90° C., for example about 30 to 80° C. The reaction pressure is not particularly limited, but may be adjusted in the range of, for example, about 0.5 to 2 atm, for example about 0.8 to 1.5 atm. According to an embodiment, the reaction time may be adjusted in the range of, for example, about 0.5 to 5 hours, for example about 1.5 to 3 hours, for example about 2 to 2.5 hours, which may be understood as illustrative.

After completion of reaction, the monobenzyltoluene isomer may be recovered by purification using a separation technique known in the art, such as extraction, distillation, crystallization, etc., alone or in combination.

According to an embodiment, as halogen (e.g., chlorine) is used in the reactant and/or catalyst, halogen (or a halogen compound) may be contained in the monobenzyltoluene product, and an example of such a halogen component may include hydrochloric acid. In cases in which the halogen component is contained in monobenzyltoluene, hydrogenation efficiency and/or dehydrogenation efficiency may decrease. Therefore, it is desirable to maintain the halogen content in monobenzyltoluene at less than predetermined level. For example, the halogen content in monobenzyltoluene may be maintained at, on an elemental basis, for example less than about 30 ppm (w/w), for example less than about 20 ppm (w/w), for example about 10 ppm (w/w). To this end, dehalogenation (for example, dechlorination) may be performed using at least one selected from among sodium methoxide (NaOCH₃), sodium carbonate (Na₂CO₃), sodium bicarbonate (NaHCO₃), and the like (purification step). As an example, the amount of the component used in dehalogenation may be set in the range of, for example, about 10 to 1000 molar equivalents, for example about 50 to 500 molar equivalents, for example about 100 to 200 molar equivalents, based on the measured halogen content, which may be understood as illustrative.

In addition, the reaction may be carried out at a temperature of, for example, about 100° C. or higher, for example about 250 to 270° C., and the reaction time is not particularly limited, but may be adjusted in the range of, for example, about 3 to 25 hours, for example about 5 to 20 hours, for example about 10 to 15 hours, which may be understood as illustrative.

According to an embodiment, monobenzyltoluene isomers may be prepared separately and combined in a predetermined ratio to obtain a monobenzyltoluene isomer mixture of desired composition. As such, the halogen content in the monobenzyltoluene isomer mixture may also be adjusted within the allowable halogen content range of the single isomer.

According to an alternative embodiment, a combination of methylbenzyl halides (i.e., 2, 3, and 4-methylbenzyl halides) corresponding to the ratio between the isomers of the monobenzyltoluene isomer mixture to be finally obtained may be used as a reactant and reacted with benzene, thereby obtaining an isomer mixture. Thereafter, the product may be purified, yielding a monobenzyltoluene isomer mixture in which the contents of individual isomers are controlled.

Meanwhile, the method described above serves to prepare monobenzyltoluene or an isomer mixture thereof by reaction of methylbenzyl halide and benzene as reactants, but in another embodiment, monobenzyltoluene or an isomer mixture thereof may be obtained by reaction of benzyl halide (e.g., benzyl chloride) and toluene as reactants. Here, as the catalyst, a heterogeneous catalyst rather than homogeneous catalyst, for example, a porous catalyst, for example a solid acid catalyst, for example a zeolite catalyst, may be used. As the zeolite catalyst, macroporous zeolite (e.g., 12-ring zeolite) may be used, and for example, any one or a combination of two or more selected from among zeolite beta, USY (ultra-stable zeolite Y), FAU (faujasite), mordenite, and the like may be used as the catalyst. Illustratively, the SAR (silica-alumina molar ratio) of this zeolite may be in the range of, for example, about 5 to 90, for example about 10 to 80, for example about 25 to 70. Also, the zeolite catalyst may be subjected to preliminary heat treatment at, for example, about 200 to 600° C., for example about 300 to 550° C., for example about 400 to 500° C. for about 0.5 to 10 hours, for example about 1 to 7 hours, example about 1.5 to 5 hours, before reaction. Thereafter, the reaction between benzyl halide and toluene may be carried out at, for example, about 25 to 200° C. (for example about 50 to 150° C., for example about 80 to 120° C.) for about 0.5 to 10 hours (for example about 1 to 6 hours, for example about 2 to 4 hours). After reaction, the catalyst may be removed from the product by solid-liquid separation known in the art (e.g., filtration, precipitation, membrane separation, centrifugation, adsorption, chromatography, ion exchange, etc.), and high-purity monobenzyltoluene or an isomer mixture thereof may be obtained using the separation and purification technique described above.

According to an embodiment of the present disclosure, among the three isomers of monobenzyltoluene, the hydrogenation/dehydrogenation rate (for example, the hydrogenation rate) of the ortho-isomer is the slowest, while the reaction rate of the para-isomer is the fastest, based on which the composition of the isomer mixture (composition) is controlled. In this regard, the activity of hydrogenation and dehydrogenation is in the order of para-MBT>meta-MBT>ortho-MBT. Accordingly, taking into consideration the tendency observed when using single isomers, it may be expected that it is preferable to use para-monobenzyltoluene alone as the LOHC, rather than using the isomer mixture, in order to maximize hydrogen storage/release efficiency.

However, the present inventors have ascertained that, when the conversion increases (e.g., about 90% or more, for example about 95% or more) in dehydrogenation using individual regioisomers, in addition to producing hydrogen-lean monobenzyltoluene, a fused hydrocarbon ring between two benzene rings is further produced, forming methylfluorene (MF) represented by Chemical Formula 1 below.

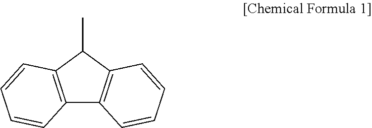

In particular, methylfluorene exhibits irreversibility during subsequent hydrogenation, which lowers reaction efficiency. During dehydrogenation, methylfluorene tends to be formed in the order of para-MBT>meta-MBT>ortho-MBT. Even when the para-monobenzyltoluene isomer is used alone, dehydrogenation selectivity decreases by the increased formation of methylfluorene during dehydrogenation due to repeated hydrogenation/dehydrogenation.

In consideration of the foregoing, in the present embodiment, the composition of the isomer mixture is primarily controlled, so that the methylfluorene content in the dehydrogenated monobenzyltoluene (MBT) isomer mixture may be maintained at, for example, less than about 10 mol %, for example less than about 7 mol %, for example less than about 5 mol %, for example less than about 3 mol %. In this regard, when the methylfluorene content in the monobenzyltoluene isomer mixture is greater than or equal to a predetermined level, hydrogenation efficiency decreases as the hydrogen storage/release cycles are repeated, which is undesirable in terms of stability. Hence, during hydrogen storage/release cycles, the methylfluorene content has to be adjusted within the range described above.

Although the present disclosure is not bound by any particular theory, it is supposed that hydrogen or an intermediate (H₆-MBT) adsorbed on the catalyst surface during dehydrogenation is involved in the formation of methylfluorene and also that methylfluorene is inevitably formed as the reaction progresses to a certain level (e.g., about 90% or more, for example about 95% or more).

Hence, in the present embodiment, rather than using a specific monobenzyltoluene isomer alone as the LOHC, a monobenzyltoluene isomer mixture is used, in which the content of para-monobenzyltoluene in the isomer mixture is adjusted, thereby improving hydrogenation/dehydrogenation efficiency and also suppressing deterioration of stability caused by methylfluorene. It is worth noting that these complex effects have not been considered in conventional techniques. In the present embodiment, the isomer mixture essentially contains para-monobenzyltoluene, but may be formulated to contain at least one of the remaining two isomers. Here, the isomer mixture may contain para-monobenzyltoluene in an amount of, for example, at least about 50 mol %, for example at least about 55 mol %, for example at least about 60 mol %. However, when the proportion of para-monobenzyltoluene increases excessively and approaches the use of a pure isomer, as described above, formation of irreversible methylfluorene also increases, so it may be advantageous to set an appropriate upper limit thereof.

In an embodiment, when para-monobenzyltoluene is combined in an appropriate ratio with one or two different regioisomers, there may be provided a balancing effect of appropriately suppressing the formation of methylfluorene while maintaining good reaction efficiency expected when para-monobenzyltoluene is used alone.

As an example, the content of ortho-monobenzyltoluene (o-MBT) in the monobenzyltoluene isomer mixture may be adjusted in the range of, for example, about 42 mol % or less, for example about 7 to 38 mol %, for example about 12 to 30 mol %, for example about 16 to 25 mol %. Furthermore, the content of meta-monobenzyltoluene (m-MBT) may be adjusted in the range of, for example, about 15 mol % or less, for example about 1 to 12 mol %, for example about 3 to 10 mol %, for example about 5 to 9 mol %. In addition, the content of para-monobenzyltoluene (p-MBT) may be adjusted in the range of, for example, about 55 to 90 mol %, for example about 60 to 85 mol %, for example about 65 to 80 mol %, for example about 70 to 75 mol %.

According to an embodiment, the monobenzyltoluene isomer mixture serving as the LOHC may exhibit a boiling point (1 atm) in the range of, for example, about 250 to 300° C. (for example about 260 to 290° C., for example about 265 to 285° C.) and a melting point of, for example, about 10° C. or less (for example about-28 to 8° C., for example about-10 to 5° C.).

Also, in an embodiment, the monobenzyltoluene isomer mixture may exhibit a viscosity (20° C.) in the range of about 3 to 8 cP (for example about 3.5 to 7.5 cP, for example about 3.8 to 6.9 cP) and a density (20° C.) of about 0.94 to 1.02 g/cm³ (for example about 0.96 to 1.00 g/cm³, for example about 0.97 to 0.99 g/cm³).

### Catalyst for Hydrogen Storage

According to an embodiment, the hydrogen storage reaction of the LOHC may be carried out in the presence of a catalyst, namely a hydrogenation catalyst (for example, a heterogeneous catalyst).

The basic composition of the hydrogenation catalyst is known in the art, and may be in the form of a hydrogenation metal supported on a support comprising a porous inorganic oxide. For example, the porous inorganic oxide may be at least one selected from the group consisting of alumina, silica, silica-alumina, aluminum phosphate, zirconia, titania, ceria, and yttria, and may be, for example, alumina.

According to an embodiment, the active metal for hydrogenation may be at least one selected from among, for example, Group IB, Group VIB, and Group VIII metals in the periodic table. For example, the active metal may be at least one selected from the group consisting of nickel, cobalt, palladium, platinum, iridium, ruthenium, rhodium, copper, gold, rhenium, zinc, molybdenum, tin, manganese, tungsten, and germanium. According to a certain embodiment, at least one selected from among palladium, platinum, iridium, ruthenium, rhodium, and the like, for example platinum, palladium, and/or ruthenium may be used as the active metal, and in a certain embodiment, ruthenium may be used.

In this regard, the content of the active metal supported on the hydrogenation catalyst may be determined in the range of, for example, about 0.1 to 50 wt %, for example about 0.5 to 30 wt %, for example about 1 to 20 wt %. As such, in order to support the active metal, any method known in the art may be adopted, such as impregnation (e.g., initial wet impregnation, excess-water impregnation, and immersion), ion exchange, coprecipitation, solid-state reaction, hydrothermal technique, sol-gel reaction, deposition-precipitation, etc., and, for example, impregnation or solid-state reaction, for example solid-state reaction, may be performed, but the present disclosure is not limited thereto.

According to an embodiment, solid-state reaction may be used when manufacturing a hydrogenation catalyst, and the active metal may be uniformly distributed on the support in a simple manner in which an active metal precursor and an inorganic oxide precursor are reacted in a single step by the addition of a base component without the use of a solvent, followed by heat treatment (for example, heat treatment under an oxygen-containing atmosphere).

In this regard, the active metal precursor may be in salt form. As an example, a nickel precursor may be at least one selected from among, for example, nickel nitrate, nickel sulfate, nickel phosphate, nickel halide, nickel carboxylate, nickel hydroxide, nickel carbonate, nickel acetylacetonate complex, nickel acetate, and hydrates thereof. A cobalt precursor may be at least one selected from the group consisting of nitrate, sulfate, carbonate, acetate, alkoxide, and halide of cobalt. Also, a palladium precursor may be at least one selected from among palladium acetate, palladium chloride, palladium nitrate, palladium sulfate, and the like. In addition, a platinum precursor may be at least one selected from among chloroplatinic acid, ammonium chloroplatinate, dinitrodiaminoplatinum, tetrachlorodiaminoplatinum, hexachlorodiaminoplatinum, dichlorodiaminoplatinum, platinum (II) dichloride, platinum (IV) tetrachloride, and the like. A ruthenium precursor may be at least one selected from among ruthenium chloride, ruthenium nitrosyl nitrate, chlorohexaaminoruthenium, and the like. Furthermore, a rhenium precursor may be at least one selected from among perrhenic acid, rhenium chloride, ammonium perrhenate, potassium perrhenate, rhenium oxide, and the like, and an iridium precursor may be at least one selected from among iridium nitrate, iridium chloride, iridium sulfate, iridium acetate, iridium acetylacetonate, iridium cyanate, iridium isopropoxide, iridium butoxide, and the like.

According to an embodiment, the precursor of the porous inorganic oxide may be an organic or inorganic acid salt of aluminum, silicon, titanium, zirconium, etc., an alkoxide thereof, a complex thereof, or a combination thereof. For example, the precursor of alumina may be at least one selected from among aluminum acetate, aluminum acetylacetonate, aluminum t-butoxide, aluminum sec-butoxide, aluminum pentoxide, aluminum ethoxide, aluminum isopropoxide, aluminum tributoxide, aluminum chloride, aluminum bromide, aluminum iodide, aluminum sulfate, aluminum nitrate, and hydrates thereof. In addition, the precursor of titania may be at least one selected from among titanium(IV) chloride, titanium(IV) bromide, titanium(IV) butoxide, titanium(IV) t-butoxide, titanium(IV) ethoxide, titanium(IV) isopropoxide, titanium(IV) bis(ethyl acetoacetate) diisopropoxide, and the like. The precursor of zirconia may be at least one selected from among zirconium acetate, zirconium(IV) acetate hydroxide, zirconium(IV) t-butoxide, zirconium(IV) hexoside, zirconium(IV) ethoxide, zirconium(IV) iodide, and the like. Also, tetraethyl orthosilicate, etc. may be used as a silica precursor.

According to an embodiment, the base component may be at least one selected from among ammonium carbonate, ammonium bicarbonate, ammonium chloride, ammonium oxalate, ammonium sulfate, ammonium hydroxide, ammonium nitrate, lithium hydroxide, sodium hydroxide, potassium hydroxide, calcium hydroxide, and magnesium hydroxide, and for example, ammonium bicarbonate may be used.

According to an embodiment, the molar ratio of active metal precursor/base component in the precursor mixture may be in the range of, for example, about 0.001 to 0.1, for example about 0.002 to 0.05, for example about 0.008 to 0.02, but may vary depending on the types of precursor and base. Also, the mixing ratio of the active metal precursor and the inorganic oxide precursor in the precursor mixture may be determined depending on desired support and metal contents in the catalyst. Solid-state reaction may be performed by applying external energy after preparation of the precursor mixture or formation of the physical mixture thereof, and the external energy applied to perform solid-state reaction between the three components described above may typically be physical or mechanical energy, for example frictional energy by milling, grinding, or pulverization, for example frictional energy by ball milling. In addition, solid-state reaction under application of external energy may be performed for, for example, about 5 to 40 minutes, for example about 10 to 30 minutes, for example about 13 to 25 minutes, which may be understood as illustrative, and the reaction time may vary depending on the reaction scale, etc.

As solid-state reaction between the precursors progresses, a gel-type catalytic solid may be ultimately obtained through processes of increasing hardness by solidification and then forming a gel again. Next, the catalytic solid formed by solid-state reaction may be heat-treated, namely fired (or calcined), under an oxygen-containing atmosphere (for example, an air atmosphere), converting the gel-type catalytic solid into an oxide. Here, heat treatment may be performed at a temperature of, for example, about 300 to 800° C., for example about 400 to 750° C., for example about 500 to 700° C. Through this heat treatment, the active metal may be evenly distributed in the pores of the inorganic oxide support. Meanwhile, the hydrogenation catalyst manufactured by heat treatment may be additionally subjected to reduction to activate the catalyst before hydrogen storage (hydrogenation). As such, hydrogen, carbon monoxide, methane, or a combination thereof may be used as a reducing gas. For example, reduction may be carried out under a hydrogen atmosphere. As an example, the reduction temperature may be in the range of, for example, about 300 to 600° C. (for example about 350 to 550° C., for example about 400 to 500° C.), and the reduction time may be determined in the range of, for example, about 1 to 10 hours, for example about 2 to 5 hours. In addition, the pressure (partial pressure of the reducing gas) during reduction is not particularly limited, but may be in the range of, for example, about 1 to 5 bar, for example about 1.5 to 4 bar, for example about 2 to 3 bar. The reduction conditions described above may be understood as illustrative and are not necessarily limited thereto.

As described above, the BET specific surface area of the support in the hydrogenation catalyst manufactured by solid-state reaction may be in the range of, for example, at least about 200 m²/g, for example at least about 300 m²/g, for example about 320 to 500 m²/g. In a certain embodiment, the BET specific surface area may be high in the range of, for example, about 350 to 480 m²/g, for example about 370 to 450 m²/g, for example about 380 to 400 m²/g. This specific surface area is much higher than that of the inorganic oxide support in a catalyst manufactured by a typical catalyst preparation method such as impregnation. Also, in an embodiment, the pore volume of the support in the catalyst may be in the range of, for example, about 0.3 to 2 cm³/g, for example about 0.4 to 1.5 cm³/g, for example about 0.6 to 1 cm³/g. Also, the average pore size (diameter) of the support in the catalyst may be in the range of, for example, about 3 to 4.5 nm, for example about 3.5 to 4.3 nm, for example about 3.8 to 4.1 nm. Also, the size of the active metal in the hydrogenation catalyst may be in the range of about 5 nm or less, for example about 3 nm or less, for example about 0.1 to 2 nm, and the active metal may be supported in the form of a single atom and/or a cluster.

According to an embodiment, the catalyst may be manufactured in the shape of a cylinder, granule, pellet, tablet, sphere, etc. To this end, molding, extrusion, spray drying, pelletization, etc. known in the art may be adopted, which may be understood as illustrative. In addition, the size (or diameter) of the catalyst manufactured in the form of a molded product may be in the range of, for example, about 0.1 to 10 mm, for example about 0.5 to 5 mm, for example about 1 to 3 mm, which may be understood as illustrative.

Although manufacturing the hydrogenation catalyst by solid-state reaction is mainly described above, the present disclosure is not limited thereto, and so long as the monobenzyltoluene isomer mixture is used as the LOHC, it may be understood that a hydrogenation catalyst manufactured based on a typical supporting method such as impregnation, etc. is also applicable. In this regard, the concentration of the active metal in the impregnation solution (in which at least one selected from the group consisting of water, ethanol, acetone, tetrahydrofuran, and hexane may be used as a solvent) may be in the range of, for example, about 0.6 to 9.1 M, for example about 1.1 to 6.6 M, for example about 3.4 to 5.8 M. The conditions for impregnation are not particularly limited, but impregnation may be carried out, for example, at about 5 to 35° C. (for example, about 20 to 25° C.) for about 6 to 24 hours (for example, about 10 to 15 hours). Such conditions may be understood as illustrative.

As described above, the active metal may be supported by impregnation on the support (e.g., a porous inorganic oxide-containing support, for example an alumina-containing support, for example a gamma-alumina support) followed by drying. For example, drying may be performed under an oxygen-containing atmosphere (for example, air), and the drying temperature may be in the range of, for example, about 40 to 150° C., for example about 90 to 110° C. Also, the drying time may be determined in the range of, for example, about 4 to 24 hours, for example about 6 to 10 hours. Thereafter, the dried catalyst may be heat-treated under the temperature conditions adopted in solid-state reaction described above. Here, heat treatment may be performed under a hydrogen-containing atmosphere (for example, hydrogen).

Meanwhile, the catalyst manufactured by impregnation may exhibit low porosity compared to the catalyst manufactured by solid-state reaction, and may have a specific surface area (BET) of, for example, about 150 to 400 m²/g, for example about 200 to 350 m²/g, for example about 250 to 300 m²/g, which is lower (e.g., about 90% or less, for example about 80% or less, for example about 70% or less) than that of the catalyst manufactured by solid-state reaction. Also, the pore volume of the catalyst manufactured by impregnation may be in the range of, for example, about 0.4 to 1 cm³/g, for example about 0.45 to 0.8 cm³/g, for example about 0.5 to 0.7 cm³/g. Also, the average pore size (diameter) thereof may be in the range of, for example, about 3 to 20 nm, for example about 5 to 12 nm, for example about 6 to 10 nm.

Meanwhile, the size of the active metal in the hydrogenation catalyst manufactured by impregnation may be in the range of about 0.4 to 10 nm, for example about 1 to 5 nm, for example about 2 to 4 nm, so the size of the supported active metal is large compared to the catalyst manufactured by solid-state reaction.

In addition, the catalyst manufactured by impregnation may be subjected to reduction before hydrogenation, and the reduction conditions may be set within the ranges described above.

In an embodiment of the present disclosure, dehydrogenation of the LOHC for hydrogen release may be performed in the of presence a catalyst, and a dehydrogenation catalyst may be of the same type as or a different type from the hydrogenation catalyst described above. Alternatively, one of the hydrogenation catalyst and the dehydrogenation catalyst may be manufactured by impregnation, and the remaining one may be manufactured by solid-state reaction, or vice versa.

### Dehydrogenation Catalyst

As such, as described above, it is desirable to suppress the formation of methylfluorene during dehydrogenation of monobenzyltoluene and to maintain the methylfluorene content in the LOHC at less than a predetermined level during repeated hydrogen storage/release cycles, and a catalyst having controlled properties may be used for dehydrogenation.

According to an embodiment, a sulfur component may be added to the catalyst to control catalytic properties, and thus, sulfidation may be performed. Here, the dehydrogenation catalyst may include a support comprising a porous inorganic oxide, an active metal, and sulfur. In the dehydrogenation catalyst, the porous inorganic oxide may be at least one selected from among the types used in connection with the hydrogenation catalyst, and the active metal may also be as described above. In the dehydrogenation catalyst according to a certain embodiment, the porous inorganic oxide support may be alumina, the active metal may be platinum, and sulfur may be incorporated into the catalyst by sulfidation.

Also, the content of the active metal in the dehydrogenation catalyst may be determined in the range of, for example, about 0.1 to 10 wt %, for example about 0.2 to 5 wt %, for example about 0.5 to 3 wt %. The sulfur content in the catalyst may be determined in the range of, for example, about 0.001 to 2 wt %, for example about 0.002 to 1 wt %, for example about 0.01 to 0.5 wt %.

In this regard, sulfidation may be performed by any method known in the art, such as a gas method (contact with hydrogen sulfide or an inert gas mixture thereof) or a liquid method (contact with a sulfur compound-containing solution). According to a certain embodiment, the heat-treated and/or reduced catalyst may be treated with a sulfur compound-containing solution.

According to an embodiment, the sulfur compound usable during sulfidation may be at least one selected from among hydrogen sulfide, hydrogen disulfide, carbon disulfide, alkyl sulfide, and the like. Examples of the alkyl sulfide may include methyl sulfide, dimethyl sulfide, dimethyl disulfide, diethyl sulfide, and/or dibutyl sulfide. In an embodiment, sulfidation may be performed at a temperature ranging from room temperature to about 500° C. (for example, about 100 to 450° C.) for about 0.5 to 100 hours (for example, about 1 to 48 hours).

According to an embodiment, the molar ratio of sulfur/active metal in the dehydrogenation catalyst may be in the range of, for example, about 0.1 to 1, for example about 0.2 to 0.8, for example about 0.3 to 0.5. If the sulfur content is too high compared to the active metal, the active site of the metal may be blocked due to the adsorption of sulfur, causing an excessive decrease in reaction activity, whereas if the sulfur content is too low, the effect of controlling the properties of the catalyst may be relatively low, so there may be limits to suppressing the formation of methylfluorene. Accordingly, the molar ratio thereof may be advantageously adjusted within the above-mentioned range, but may vary depending on the types of active metal and support, the amount of active metal, and the particle size. In addition, the degree of sulfidation by sulfidation may be adjusted in the range of, for example, about 10 to 70%, for example about 20 to 60%, for example about 30 to 50%.

In an alternative embodiment, when the dehydrogenation catalyst is manufactured by solid-state reaction, an effect equivalent to sulfidation may be achieved by performing solid-state reaction by addition of a sulfur compound (e.g., at least one selected from among ammonium sulfate, hydroxylamine sulfate, aluminum sulfate, dimethyl sulfate, diethyl sulfate, tetramethyl ammonium sulfate, tetrabutyl ammonium sulfate, ammonium dodecyl sulfate, and the like) along with the active metal precursor, the inorganic oxide precursor, and the base component in the precursor mixture, and the amount of the sulfur compound may be determined in the range described in relation to sulfidation.

In the dehydrogenation catalyst having controlled properties in this way, it is supposed that the formation of methylfluorene may be more effectively suppressed by weakening the adsorption strength of the dehydrogenation product and preventing additional reaction from proceeding after H₀-MBT.

### Hydrogenation/Dehydrogenation Using Monobenzyltoluene Isomer Mixture

According to an embodiment of the present disclosure, hydrogenation/dehydrogenation may be performed using the monobenzyltoluene isomer mixture described above as the liquid organic hydrogen carrier (LOHC). As such, monobenzyltoluene serves to store or release hydrogen in a manner that alternates between a hydrogen-rich state and a hydrogen-lean state in liquid or gas phase. In particular, by handling hydrogen in the form of a liquid organic hydrogen carrier, existing fuel facilities may be utilized, and hydrogen with high purity may be produced compared to hydrogen storage by hydrogenation of gas (e.g., carbon dioxide or nitrogen).

The method of storing and releasing hydrogen according to an embodiment may comprise bringing a monobenzyltoluene isomer mixture (for example in liquid form) as a liquid organic hydrogen carrier into contact with a hydrogenation catalyst or a dehydrogenation catalyst in a reaction system. In an embodiment, the ratio of active metal in the catalyst/LOHC (M/R) may be distinguished between hydrogenation and dehydrogenation, and for example, the ratio of M/R may be set relatively low in hydrogenation, while the ratio of M/R may be set relatively high in dehydrogenation. In this regard, if the ratio of M/R is too low, insufficient reaction activity may be exhibited because reaction progression is limited due to a lack of active sites of the active metal. On the other hand, if the ratio of M/R is too high, the volume of the solid catalyst may increase significantly compared to the liquid reactant, forming an excessive slurry state, which not only makes it difficult to perform stirring during reaction, but also creates a diffusion limitation, resulting in a phenomenon in which the active sites of the catalyst cannot be sufficiently utilized. Hence, it may be advantageous to appropriately adjust the ratio of M/R within the above-mentioned range.

In an embodiment, the ratio of M/R in hydrogenation (hydrogen storage) may be adjusted in the range of, for example, about 0.01 to 0.4 mol % (for example about 0.02 to 0.2 mol %, for example about 0.03 to 0.15 mol %, for example about 0.035 to 0.1 mol %), whereas the ratio of M/R in dehydrogenation (hydrogen release) may be adjusted in the range of, for example, about 0.3 to 3 mol % (for example about 0.4 to 2.5 mol %, for example about 0.5 to 2 mol %, for example about 0.55 to 1 mol %).

According to an embodiment, the hydrogenation temperature may be in the range of, for example, about 50 to 200° C., for example about 70 to 180° C., for example about 80 to 150° C. Also, the hydrogenation pressure may be in the range of, for example, about 5 to 100 bar, for example about 20 to 80 bar, for example about 30 to 70 bar, for example about 40 to 60 bar. In addition, the hydrogenation time is not particularly limited, but may be in the range of, for example, about 0.5 to 24 hours, for example about 1 to 12 hours, for example 1.5 to 6 hours.

According to an embodiment, hydrogenation efficiency (defined as the number of moles of hydrogen actually stored by hydrogenation relative to the number of moles of hydrogen that may theoretically be stored in 1 mole of dehydrogenation reactant) may be, for example, at least about 90%, for example at least about 95%, for example at least about 99%. As such, selectivity for the hydrogenation product corresponding to the hydrogen storage compound may be, for example, at least about 90%, for example at least about 95%, for example at least about 99%.

On the other hand, dehydrogenation may be performed to release hydrogen from the hydrogen storage material (liquid organic hydrogen carrier). Dehydrogenation, which is endothermic, may be performed at a reaction temperature higher (by, for example, about 50 to 150° C., for example about 60 to 120° C., for example about 80 to 100° C.) than hydrogenation (exothermic) of the same hydrogen storage material. According to an embodiment, the dehydrogenation temperature may be in the range of, for example, about 100 to 300° C., for example about 150 to 290° C., for example about 180 to 280° C. Also, unlike the reaction temperature, the dehydrogenation pressure may be, for example, about 0.1 to 10 bar, for example about 0.5 to 5 bar, for example atmospheric pressure, which is low compared to the pressure conditions of hydrogenation. Also, the dehydrogenation time is not particularly limited, but may be set in the range of, for example, about 0.5 to 24 hours, for example about 2 to 12 hours, for example about 3 to 8 hours.

According to an embodiment, dehydrogenation efficiency (defined as the number of moles of hydrogen actually produced by dehydrogenation relative to the number of moles of hydrogen that may theoretically be released from 1 mole of the hydrogenation reactant) may be, for example, at least about 50%, for example at least about 60%, for example at least about 70%, for example at least about 80%, and in a certain embodiment, may be as high as, for example, at least about 90%, for example at least about 95%. As such, selectivity for the dehydrogenation product corresponding to the hydrogen storage compound may be, for example, at least about 40%, for example at least about 70%, for example at least about 80%.

Meanwhile, according to an embodiment, hydrogenation/dehydrogenation may be performed using a reactor known in the art, such as a batch reactor, a continuous reactor, a semi-continuous reactor, etc., and the reactor may be provided with a stirrer because hydrogenation and dehydrogenation may occur in a liquid phase. According to an embodiment, the reaction may be performed in a fixed-bed reactor using a catalyst in the form of particles.

### MODE FOR INVENTION

The present disclosure may be more clearly understood by the following examples, which are merely set forth to illustrate the present disclosure and are not to be construed as limiting the scope of the present disclosure.

### EXAMPLES

In Examples and Comparative Examples, the prepared catalysts were analyzed according to the following analysis methods.

### GC-FID (Gas Chromatography-Flame Ionization Detector) Analysis

The sample recovered after reaction was filtered through a 0.1 μm syringe filter, and 1 g of the corresponding material was dissolved in 10 ml of acetone and 100 μl of nonane to prepare a sample for GC-FID analysis. GC-FID analysis was performed on a 7890A from Agilent Technologies with a Restek Rxi®-17Sil MS column (30 m×0.25 mm×0.25 μm) connected thereto. For GC-FID analysis, inlet temperature=300° C., detector temperature=300° C., flow rate=0.2 cm³/min, split ratio=1:300, and the oven program was set in 50° C.→heating at 50° C./min→maintenance at 140° C. for 80 minutes→heating at 50° C./min→maintenance at 250° C.

¹H NMR and ¹³C NMR Analysis

NMR analysis was performed using DPX-300 from Bruker. All NMR spectra were acquired in CDCl₃, and tetramethylsilane was used as a reference.

### Analysis of Adsorption Energy Based on DFT (Density Functional Theory)

DFT calculations were performed using the Vienna ab initio simulation package (VASP). The generalized gradient approximation of Perdew-Burke-Ernzerhof was adopted as the exchange correlation function. In addition, the projector-augmented wave potential was used along with a regular wave set for analogy of the valence wave function. The cut-off energy for all DFT calculations was set at 500 eV. The conjugate gradient algorithm was used for all geometric optimizations until the force acting on each atom converged to within 0.03 eV/Å. The electronic work function was determined according to the Methfessel-Paxton method with an energy spread of 0.2 eV. To consider the dispersion force effect, the semiempirical DFT-D3 method was applied. The Monkhorst-Pack Brillouin zone sampling was 3×3×1 (9×9×9) for the Pt and Ru slab models and 2×2×1 (6×4×4) for the γ-Al₂O₃ slab model. Meanwhile, the slab model of each of the Pt(111) (5×4) supercell with 3 layers (2 layers relaxed), the Ru(0001) (6×4) supercell with 3 layers (2 layers relaxed), and the γ-Al₂O₃(110) (2×2) supercell with 4 layers (1 layer relaxed) was applied.

### Analysis of Concentration of Chlorine

The synthesized material was subjected to KS M 2457:2003 test method (method for testing chlorine content in light and heavy distillates by oxidative microcoulometry). The content of chlorine component was analyzed as follows using a TS and TX analyzer (ECS3000) from Thermo Fisher Scientific by oxidative microcoulometry according to ASTM D5808. Oxygen and inert gas are continuously injected into a combustion tube to combust the sample, and the resulting combustion product is transferred to a titration cell for reaction with silver ions generated by electrolysis and hydrochloric acid, after which the silver ions consumed by the titration reaction are coulometrically replaced, and the total current required to replace the silver ions is thus a measure of chlorine.

### Analysis of Boiling Point, Melting Point, and Viscosity

The boiling point was measured as follows: 1 ml of a liquid sample was added to a test tube, and a melting point capillary tube was dropped into the test tube with the open end facing down. The test tube was attached to the thermometer using a rubber band and placed inside a TL tube filled with heat medium oil. Next, the TL tube was heated using a Bunsen burner, and the temperature was raised until bubbles were continuously generated in the capillary tube. After the Bunsen burner was turned off, gas bubbling slowed and stopped. The boiling point was recorded as the temperature at which the substrate began to enter the capillary tube. Measurement was performed three times on each material, and the average value was used.

Melting point was determined using data stored in Aspen+ software.

Viscosity was measured using a DV-II+Pro viscometer from Brookfield Engineering Laboratories. For analysis, 6 ml of a liquid sample was placed on a sample cup and the shear rate was changed from 100 s⁻¹ to 200 s⁻¹. Measurement was performed for 2 minutes at each temperature.

### Thermogravimetric Analysis

For thermogravimetric analysis, each of the three monobenzyltoluene isomers (5 ml) and a γ-Al₂O₃ or Ru/Al₂O₃ catalyst (500 mg) were stirred at room temperature for 24 hours and then dried under vacuum at 80° C. for 12 hours, whereby each sample adsorbed with monobenzyltoluene was prepared. The sample thus prepared was subjected to thermogravimetric analysis using SDT Q600 from TA Instruments. Thermogravimetric analysis was performed by placing 5 mg of the sample adsorbed with monobenzyltoluene on an alumina pan and heat-treating the same from 35° C. to 600° C. at a heating rate of 5° C./min under nitrogen flow (100 sccm).

### Examples

Preparation of Hydrogenation Catalyst (Ru/Al₂O₃)

Al₂O₃ was added to a solution of Ru₃(CO)₁₂ (purity: 99%, Sigma-Aldrich) dissolved in tetrahydrofuran (purity: 99.5%; Daejung Chemicals & Metals) and stirred at 25° C. for 13 hours. Then, the solvent was removed at 45° C. under reduced pressure followed by drying in an oven at 105° C. for 8 hours. Finally, the recovered sample was heat-treated at 500° C. for 6 hours under hydrogen flow (100 sccm), thereby producing a hydrogenation catalyst (Ru/Al₂O₃).

Preparation of Dehydrogenation Catalyst (Pt—S/Al₂O₃)

Aluminum nitrate hydrate (purity: 98.0%; Junsei), ammonium bicarbonate (purity: 97.0%; Daejung Chemicals & Metals), ammonium sulfate (purity: 99.0%; Sigma-Aldrich), and [Pt(NH₃)₄](NO₃)₂ (purity: 99.0%; Alfa Aesar) were pulverized (or milled) with a mortar and pestle for 20 minutes to prepare a gel-type catalytic solid. Then, the gel-type catalytic solid was fired at 600° C. for 5 hours under an oxygen-containing atmosphere (for example, an air atmosphere). Finally, the recovered sample was heat-treated at 500° C. for 3 hours under hydrogen flow (100 sccm), thereby producing a dehydrogenation catalyst (Pt—S/Al₂O₃).

### Synthesis of Single Isomers of Monobenzyltoluene

Referring to FIG. 1, three monobenzyltoluene isomers (o-MBT, m-MBT, and p-MBT) were synthesized from 2, 3, and 4-methylbenzyl chlorides and excess benzene in the presence of a homogeneous catalyst.

Specifically, 2-methylbenzyl halide, 3-methylbenzyl halide, and 4-methylbenzyl halide (purity: >98.0%; TCI) were prepared, and each was added to a stirrer along with 20 molar equivalents of benzene (purity: 99.5%; Duksan Chemical) and 0.01 molar equivalents of FeCl₃ (purity: 99.5%; Alfa Aesar). The added mixture was reacted with stirring (800 rpm) at room temperature for 2 hours, and after reaction, distilled water was added and stirred at 1000 rpm for 10 minutes to deactivate the catalyst. Next, ethyl acetate (purity: 99.0%; Samchun Chemicals) was added and an extraction process was performed to separate the aqueous solution layer and the organic layer, obtaining only the organic layer. Additionally, excess brine and excess MgSO₄ (purity: 99.5%; Samchun Chemicals) were added and an extraction process was performed with ethyl acetate to remove as much water as possible. Finally, the recovered organic product was purified by a vacuum distillation process, thereby recovering o-MBT, m-MBT, and p-MBT. As such, the yield was about 40 to 48% of the theoretical yield.

When removing the trace amount of chlorine contained in each of the three MBT isomers as products, 200 molar equivalents of sodium methoxide (purity: 95%; Sigma-Aldrich) for a purification process were added based on the measured chlorine content. The added mixture was heated to 270° C. with stirring (750 rpm) while supplying nitrogen gas at 0.03 L/min, maintained for 12 hours, and then cooled. Next, distillation was performed under reduced pressure (vacuum) of 0.85 bar, and materials with a boiling point in the range of 250 to 260° C. were recovered, obtaining chlorine-free three monobenzyltoluene isomers.

### Preparation of Monobenzyltoluene Isomer Mixtures

A H₀-MBT-p55 isomer mixture (ortho:meta:para=36:9:55) and a H₀-MBT-p72 isomer mixture (ortho:meta:para=19:9:72) with increased p-MBT content were prepared according to the following procedures.

Toluene (purity: 99.8%; Samchun Chemicals) and benzyl chloride (purity: 99.0%; Samchun Chemicals) were prepared as reactants. Also, beta zeolite (Zeolyst Co.) and USY zeolite (Zeolyst Co.) were prepared as catalysts followed by heat treatment at 450° C. for 2 hours.

First, toluene and the catalyst were added to a reactor and heated to the set reaction temperature. Then, an appropriate amount of benzyl chloride was added depending on the amount of toluene and reacted together with stirring (1000 rpm) at 110° C. for 3 hours. After reaction, the solid catalyst was separated from the mixture followed by additional distillation, thereby obtaining high-purity monobenzyltoluene.

In addition, an MBT isomer mixture (MLH-sim) was prepared as a comparative example by physically mixing the three single isomers to correspond to the composition of Marlotherm LH commercially available from Sasol (ortho:meta:para=46:5:49).

### Analysis Results

### Characterization of Single Isomers of Monobenzyltoluene

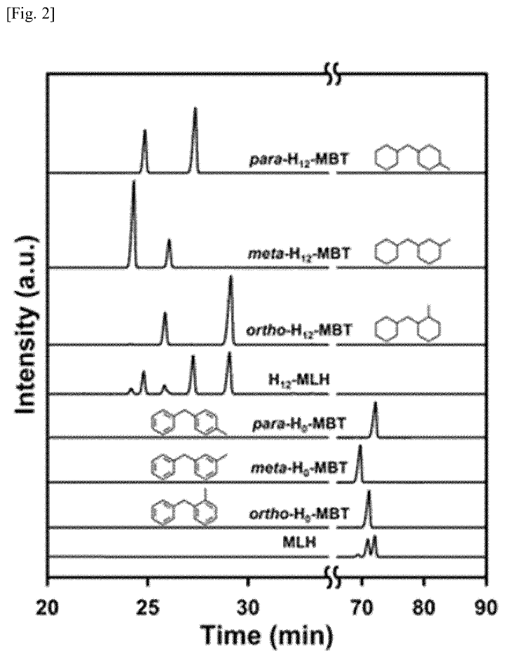

The results of GC analysis of the three single isomers of monobenzyltoluene (o-MBT, m-MBT, and p-MBT) in the hydrogen-rich (H₁₂-MBT) and hydrogen-lean (H₀-MBT) states of monobenzyltoluene and the isomer mixture (comparative example) corresponding to commercially available Marlotherm LH are shown in FIG. 2. Referring thereto, the hydrogen-lean state appeared to have one peak per isomer, and the hydrogen-rich state appeared to have two peaks per isomer. These correspond to the peaks of commercial Marlotherm LH, and in particular, one peak in each of the hydrogen-rich ortho and meta isomers was detected in almost the same region and appeared to overlap.

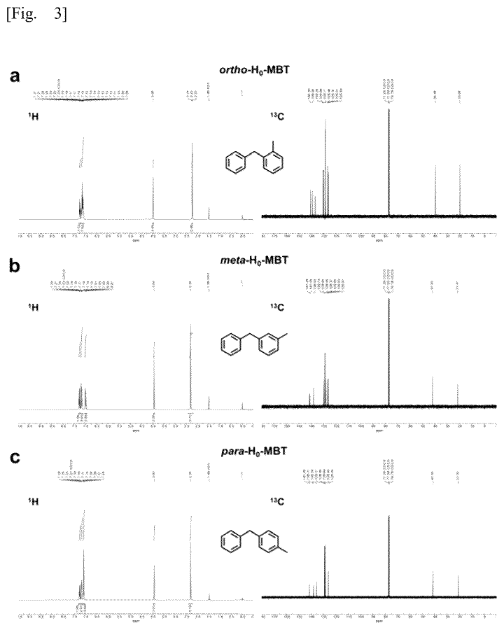

¹H NMR (left) and ¹³C NMR (right) were performed on three single isomers of monobenzyltoluene (o-MBT, m-MBT, and p-MBT), and the analysis results thereof are shown in FIGS. 3a to 3c. Referring thereto, in the single isomers of monobenzyltoluene, the methyl group, which is a substituent, was present in the ortho, meta, and para positions, confirming that each material was an isomer of monobenzyltoluene.

### Analysis of Effect of Chlorine in Single Isomers of Monobenzyltoluene

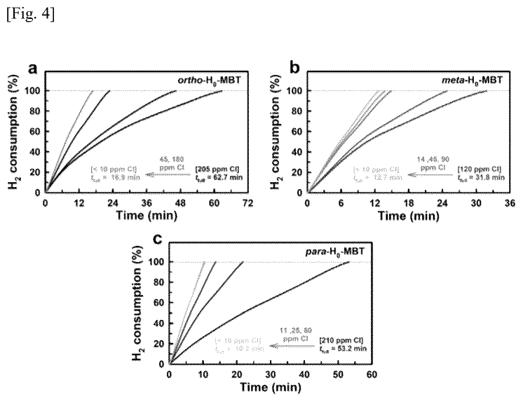

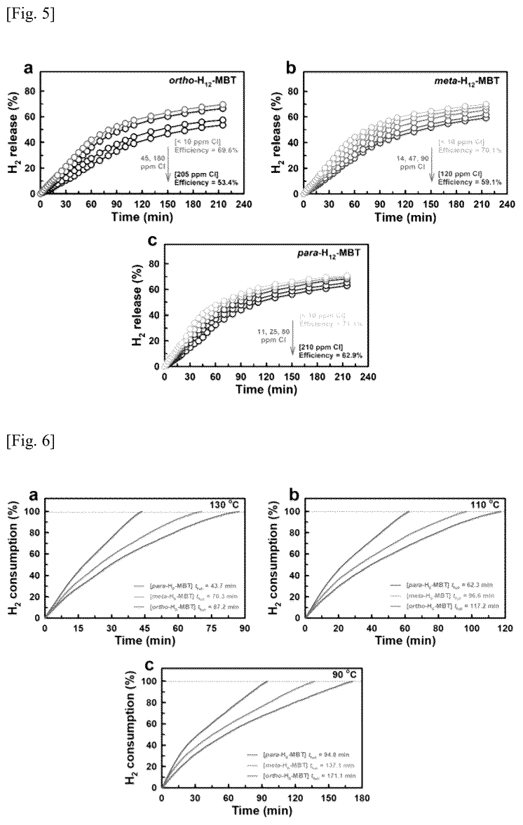

The results of hydrogenation and dehydrogenation of the three single isomers of monobenzyltoluene (o-MBT, m-MBT, and p-MBT) depending on the concentration of Cl are shown in Table 1 below and FIGS. 4 and 5.

TABLE 1

| | | Amount of | H₁₂-BT | H₀-BT | H₂ release |
|---|---|---|---|---|---|
| Classi- | | remaining | conversion | selectivity | efficiency |
| fication | Material | Cl [ppm] | [mol %]^{a} | [mol %]^{a} | [mol %]^{b} |
| Example 1 | ortho- | 205 | 73.4 | 42.1 | 52.4 |
| | H₁₂-MBT | 180 | 80.2 | 41.6 | 57.4 |
| | | 45 | 85.5 | 53.2 | 66.4 |
| | | <10 | 86.8 | 59.4 | 69.6 |
| Example 2 | meta- | 120 | 81.0 | 43.5 | 59.1 |
| | H₁₂-MBT | 90 | 83.6 | 46.3 | 61.8 |
| | | 47 | 84.4 | 52.7 | 65.4 |
| | | 14 | 86.4 | 56.0 | 68.0 |
| | | <10 | 87.3 | 59.8 | 70.1 |
| Example 3 | para- | 210 | 80.8 | 53.6 | 62.9 |
| | H₁₂-MBT | 80 | 84.1 | 55.4 | 65.3 |
| | | 25 | 86.9 | 56.7 | 68.2 |
| | | 11 | 88.1 | 58.4 | 69.6 |
| | | <10 | 88.3 | 59.9 | 71.1 |

^{a}Calculation result by GC analysis.
^{b}Hydrogen release efficiency = (full dehydrogenation product [mol] × 6H₂ + single-ring dehydrogenation product [mol] × 3H₂)/(reactant [mol] × 6H₂) × 100%

Referring to the table and drawings, as the concentration of Cl in each isomer decreased, the time (t_{full}) required for hydrogenation to be completed during hydrogenation decreased, confirming increased reaction activity. In addition, similar to hydrogenation, the hydrogen (H₂) release efficiency was found to increase with a decrease in the concentration of Cl during dehydrogenation. As such, the activity increased in both hydrogenation and dehydrogenation until the concentration of Cl remaining in monobenzyltoluene was lowered to less than 10 ppm.

The boiling point, melting point, and viscosity of three single isomers of monobenzyltoluene (o-MBT, m-MBT, and p-MBT) subjected to purification by dechlorination were compared with commercial Marlotherm LH corresponding to the comparative example, and are given in Table 2 below.

TABLE 2

| | | Boiling | Melting | |
|---|---|---|---|---|
| | | point | point | Viscosity |
| Classification | Material | [° C.]^{a} | [° C.]^{b} | [cP]^{c} |
| Example 1 | ortho-H₀-MBT | 284.7 | 6.6 | 5.26 |
| Example 2 | meta-H₀-MBT | 278.3 | −27.8 | 6.41 |
| Example 3 | para-H₀-MBT | 288.3 | 4.6 | 5.19 |
| Example 4 | ortho-H₁₂-MBT | 261.3 | — | 6.67 |
| Example 5 | meta-H₁₂-MBT | 266.3 | — | 4.92 |
| Example 6 | para-H₁₂-MBT | 272.0 | — | 5.62 |
| Comparative | Marlotherm LH | 282.7 | — | 4.36 |
| Example 1 | | | | |
| Comparative | Marlotherm H₁₂-LH | 271.0 | — | 6.59 |
| Example 2 | | | | |

^{a}Result measured by experiment.
^{b}Based on Aspen+ software data.
^{c}Result measured at 20° C. using DV-II + Pro viscometer.

As is apparent from the table above, the hydrogen-lean isomer had a higher boiling point than the hydrogen-rich isomer, and in particular, the para-isomer exhibited a higher boiling point than the other isomers. For melting point, since it was difficult to measure, data from the ASPEN+ program was cited, and the hydrogen-lean meta-isomer exhibited a particularly low melting point. In addition, the viscosity tended to decrease with an increase in the temperature, and the viscosity of the synthesized isomers was measured to be about 4.92 to 6.67 cP at 20° C.

### Evaluation of Activity of Single Isomers of Monobenzyltoluene and Physical Mixtures Thereof

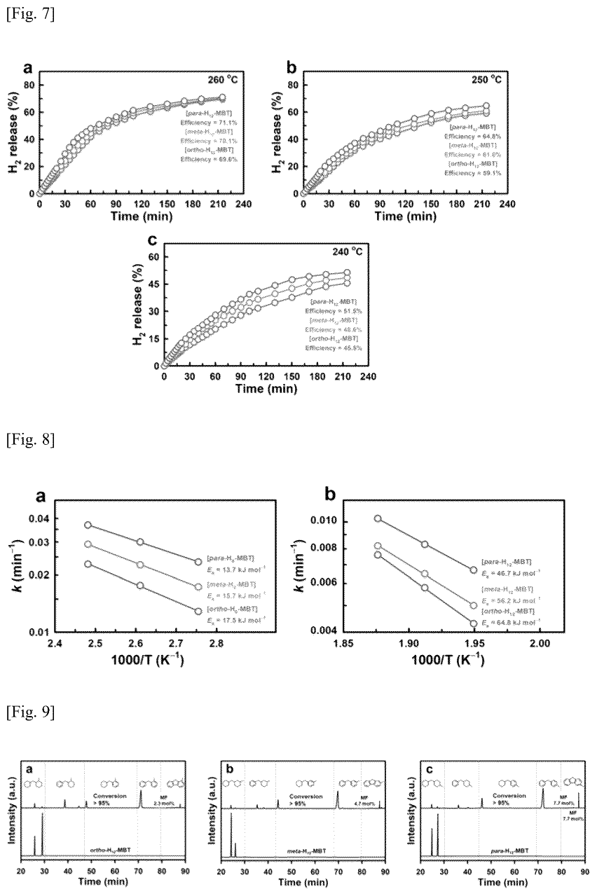

The results of hydrogenation and dehydrogenation performed after removing Cl from the three monobenzyltoluene isomers (o-MBT, m-MBT, and p-MBT) are shown in FIGS. 6 and 7. FIGS. 6a to 6c show results of hydrogenation of purified monobenzyltoluene at respective reaction temperatures of 130° C., 110° C., and 90° C. These drawings show the H₂ consumption efficiency over time. Based on the value of t_{full}, the hydrogenation activity was in the order of ortho<meta<para-monobenzyltoluene. FIGS. 7a to 7c show results of dehydrogenation of purified monobenzyltoluene at respective reaction temperatures of 260° C., 250° C., and 240° C. These drawings show the H₂ release efficiency over time, and the dehydrogenation activity was also in the order of ortho<meta<para-monobenzyltoluene, similar to hydrogenation.

Arrhenius plots of hydrogenation and dehydrogenation performed after removing Cl from the three monobenzyltoluene isomers (o-MBT, m-MBT, and p-MBT) are shown in FIGS. 8a and 8b, respectively. In FIG. 8a, the Arrhenius plots of hydrogenation were depicted based on the initial reaction rate constants calculated at 90° C., 110° C., and 130° C., and the activation energy was also in the order of ortho>meta>para-monobenzyltoluene, indicating a tendency consistent with the reaction activity. In FIG. 8b, the Arrhenius plots of dehydrogenation were depicted based on the initial reaction rate constants calculated at 240° C., 250° C., and 260° C., and the activation energy was consistent with the tendency in hydrogenation.

The results of GC-FID analysis after dehydrogenation performed after removing Cl from the three monobenzyltoluene isomers (o-MBT, m-MBT, and p-MBT) are shown in FIGS. 9a to 9c. Referring thereto, when the H₁₂-MBT conversion exceeded 95%, new peaks were observed after 80 minutes. Based on the results of GC-MS analysis, the components corresponding to the new peaks were found to be 1-, 2-, and 3-methylfluorene (MF). Based on the results of GC analysis, the amount of methylfluorene formed was in the order of para>meta>ortho-monobenzyltoluene, similar to the hydrogenation and dehydrogenation activities. Although the use of the single para-monobenzyltoluene isomer seems to be preferable to maximize hydrogen storage/release efficiency, the amount of methylfluorene formed also increased, confirming decreased dehydrogenation selectivity.

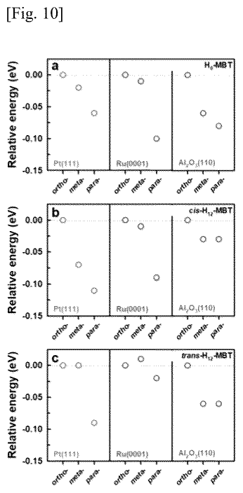

In order to analyze the difference in hydrogen charging and discharging rates depending on the type of isomer, the adsorption energies for Pt(111), Ru(0001), and γ-Al₂O₃ (110) sites of H₀-MBT, cis-H₁₂-MBT, and trans-H₁₂-MBT were calculated based on the density functional theory (DFT), and the results thereof are shown in FIGS. 10a to 10c. Referring thereto, H₀-MBT, cis-H₁₂-MBT, and trans-H₁₂-MBT all had the lowest adsorption energy for the para-isomer, indicating that the adsorption strength was the strongest.

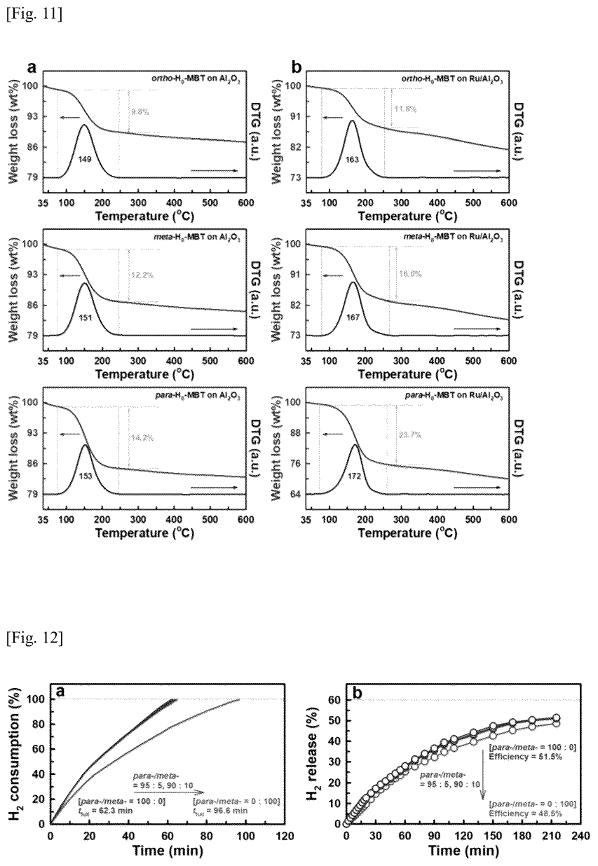

Each of the three monobenzyltoluene isomers (hydrogen-lean state) was adsorbed on gamma-alumina (γ-Al₂O₃) and Ru/Al₂O₃ catalysts and thermogravimetric analysis was performed, and the results thereof are shown in FIGS. 11a to 11c. Referring thereto, the para-isomer exhibited the lowest adsorption energy for all sites and the strongest adsorption strength. On the other hand, the ortho-isomer was evaluated to be the most unstable during adsorption and thus have weak adsorption strength. In FIG. 11a, the adsorption strength and amount on gamma-alumina were all in the order of ortho<meta<para-monobenzyltoluene. In FIG. 11b, the adsorption strength and amount on the commercial Ru/Al₂O₃ catalyst are represented, indicating a similar tendency as in the case of gamma-alumina. In short, the adsorption strength was in the order of para>meta>ortho-monobenzyltoluene, which is supposed to be because the difference in initial reaction rate was caused by the adsorption strength and amount, resulting in the hydrogenation tendency described above.

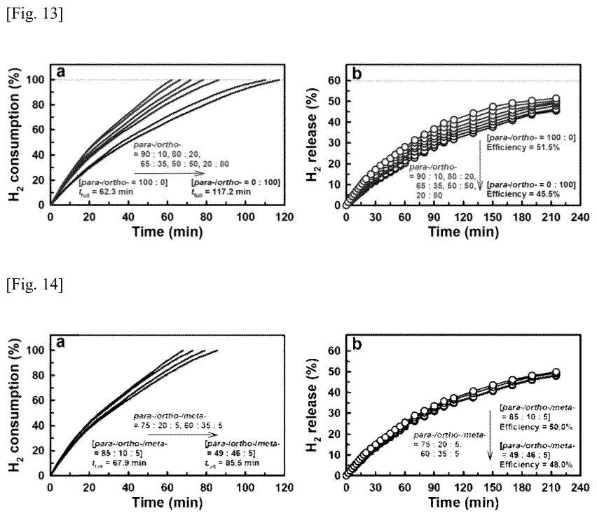

The physical mixture of monobenzyltoluene isomers was subjected to hydrogenation and dehydrogenation, and the results thereof are shown in FIGS. 12 to 14. In this regard, the results of hydrogenation and dehydrogenation of the binary physical mixture of two monobenzyltoluene isomers (para and meta) and the binary physical mixture of two monobenzyltoluene isomers (para and ortho) are shown in FIGS. 12 and 13, respectively, and the results of hydrogenation and dehydrogenation of the ternary physical mixture of three monobenzyltoluene isomers (ortho, meta, and para) are shown in FIG. 14. Referring thereto, the activity of single isomers of monobenzyltoluene appeared in the order of ortho<meta<para-monobenzyltoluene, and as the composition of the isomer with good activity increased, the corresponding reaction activity also increased. Furthermore, as shown in FIG. 12, when the amount of the para-isomer was 90 mol % or more, the amount of the meta-isomer did not have a significant effect on the hydrogenation and dehydrogenation results. On the other hand, as shown in FIG. 13, the ortho-isomer had a greater impact on the hydrogenation and dehydrogenation results than the meta-isomer. Therefore, it is supposed to be desirable to lower the amount of ortho-monobenzyltoluene during hydrogenation/dehydrogenation.

### Evaluation of Activity and Stability of Selective Isomer Compositions

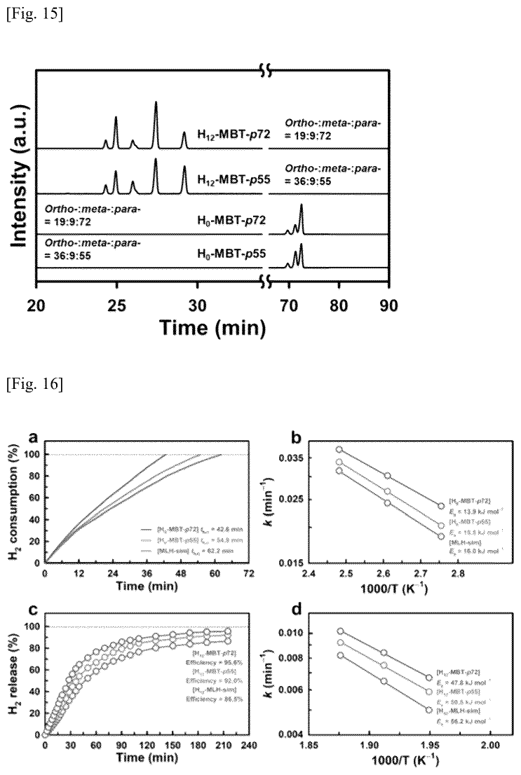

GC analysis was performed in the hydrogen-rich state (H₁₂-MBT) and the hydrogen-lean state (H₀-MBT) of selective isomer compositions (p72 and p55) obtained by a synthesis process of increasing the composition of para-monobenzyltoluene. The physical properties and GC analysis results of the isomer compositions are shown in Table 3 below and FIG. 15.

TABLE 3

| | | Boiling | Melting | |
|---|---|---|---|---|
| | | point | point | Viscosity |
| Classification | Material | [° C.]^{a} | [° C.]^{b} | [cP]^{c} |
| Example 1 | H₀-MBT-p55 | 282.9 | — | 4.08 |
| Example 2 | H₀-MBT-p72 | 282.9 | −5.2 | 3.87 |
| Example 3 | H₁₂-MBT-p55 | 265.0 | — | 6.92 |
| Example 4 | H₁₂-MBT-p72 | 266.0 | — | 6.73 |
| Comparative | SIM H₀-LH | 282.8 | — | 4.15 |
| Example 1 | | | | |
| Comparative | SIM H₁₂-LH | 266.9 | — | 7.15 |
| Example 2 | | | | |

^{a}Result measured by experiment.
^{b}Based on Aspen+ software data.
^{c}Result measured at 20° C. using DV-II + Pro viscometer.

As shown in the drawing, when the selective isomer compositions were converted from the hydrogen-lean state into the hydrogen-rich state by hydrogenation, the composition of ortho-, meta-, and para-monobenzyltoluene did not change.

The results of hydrogen storage and release of selective isomer compositions (p72 and p55) obtained by a synthesis process of increasing the composition of para-monobenzyltoluene and the comparative example MLH-sim are shown in FIGS. 16a and 16c, and also Arrhenius plots of hydrogenation and dehydrogenation are shown in FIGS. 16b and 16d. Referring thereto, Arrhenius plots were created based on the initial reaction rate constants at 90° C., 110° C., and 130° C. for hydrogenation and at 240° C., 250° C., and 260° C. for dehydrogenation. As the amount of para-monobenzyltoluene increased, the reaction activity of individual compositions also increased, and was evaluated in the order of MBT-p72>MBT-p55>MLH-sim. Also, the activation energy was in the order of MBT-p72<MBT-p55<MLH-sim.

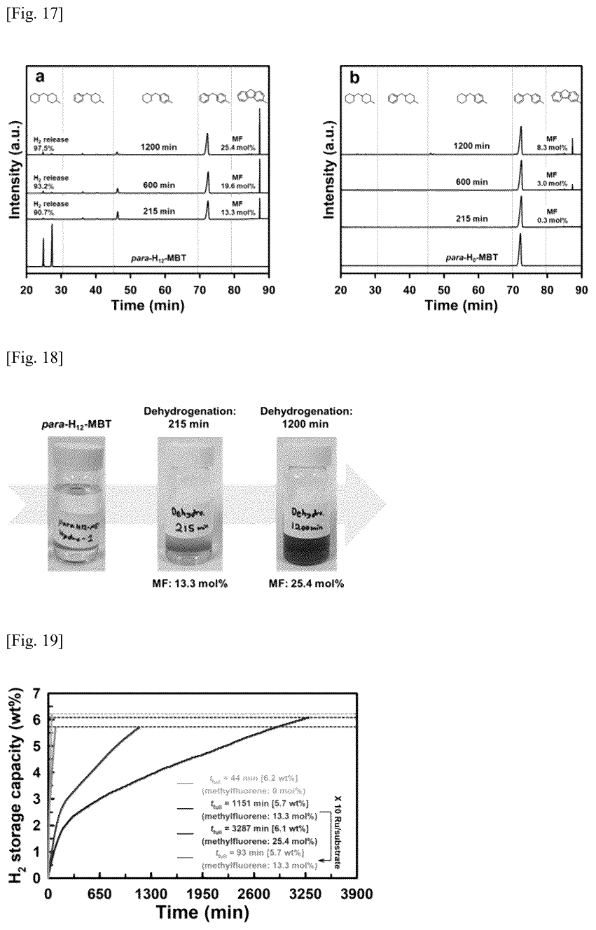

GC analysis of the products obtained by dehydrogenating para-monobenzyltoluene was performed, and the results thereof are shown in FIGS. 17a and 17b. As such, FIG. 17a corresponds to the hydrogen-rich state (p-H₁₂-MBT), and FIG. 17b corresponds to the hydrogen-lean state (p-H₀-MBT). Referring to FIG. 17a, as the dehydrogenation time of p-H₁₂-MBT increased, the molar concentration of methylfluorene increased to 13.3 mol %, 19.6 mol %, and 25.4 mol %. However, when p-H₀-MBT as the reactant was used, a very small amount of methylfluorene was formed in the same reaction time. From the above-mentioned results, it was confirmed that hydrogen or the intermediate (H₆-MBT) adsorbed on the catalyst surface was involved in the formation of methylfluorene and also that methylfluorene was inevitably formed as the reaction progressed further under a situation where the reaction proceeded 90% or more.

The apparent colors of para-monobenzyltoluene and products obtained by dehydrogenation for 215 minutes and 1200 minutes using the same as the reactant are shown in FIG. 18. Referring thereto, based on results of comparison of the apparent colors of para-monobenzyltoluene before reaction and dehydrogenation products thereof, it was confirmed that para-monobenzyltoluene was colorless in the hydrogen-rich state and then gradually changed from yellow to reddish-yellow as methylfluorene (MF) was formed by dehydrogenation.

Meanwhile, when hydrogenation was performed again after dehydrogenation of para-monobenzyltoluene, the effect of the concentration of methylfluorene (MF) in the dehydrogenation product on hydrogenation was evaluated, and the results thereof are shown in FIG. 19. Referring thereto, when the concentration of methylfluorene was 13.3 mol % and 25.4 mol % compared to pure p-H₀-MBT, hydrogenation activity decreased to the extent that the time (t_{full}) required for hydrogen storage (charging) increased approximately 30 to 90 times. However, for the reactant in which the concentration of methylfluorene was 13.3 mol %, the t_{full} value of 93 minutes was obtained by the process of adding 10 times the commercial Ru/Al₂O₃ catalyst. In short, when the H₁₂-MBT conversion of 90% or more is achieved by increasing dehydrogenation efficiency, methylfluorene is formed, and methylfluorene has a significantly negative effect on hydrogenation at a level greater than or equal to a predetermined concentration, so efficient hydrogen storage/release cannot be realized.

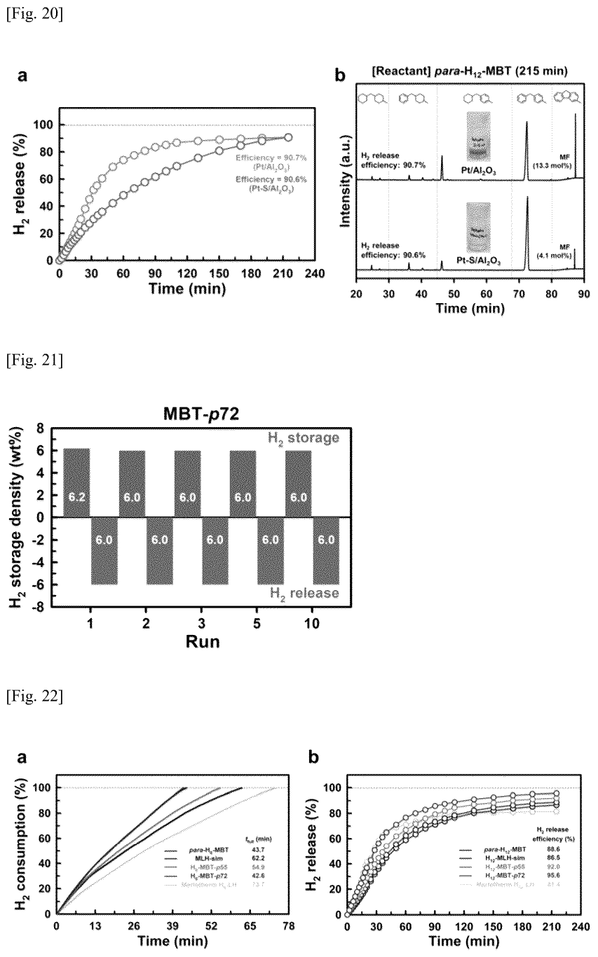

Dehydrogenation of para-monobenzyltoluene in the presence of each of the Pt/Al₂O₃ catalyst and the Pt—S/Al₂O₃ catalyst and GC analysis of the obtained products were performed, and the results thereof are shown in FIGS. 20a and 20b. Referring thereto, when using the Pt—S/Al₂O₃ catalyst, the amount of methylfluorene formed under conditions of the same reaction time and the same hydrogen release efficiency (90%) decreased from 13.3 mol % to 4.1 mol %, compared to when using the Pt/Al₂O₃ catalyst.

Therefore, in order to utilize monobenzyltoluene as a reversible and efficient hydrogen storage material, it is desirable to lower the amount of methylfluorene formed during dehydrogenation as much as possible. To this end, the use of the dehydrogenation catalyst having controlled properties was confirmed to be effective.

The stability of the selective isomer composition (p72) obtained by a synthesis process of increasing the content of para-monobenzyltoluene was evaluated using the Pt—S/Al₂O₃ catalyst instead of the Pt/Al₂O₃ catalyst during dehydrogenation. To this end, hydrogenation-dehydrogenation cycles were repeated. Here, in order to suppress the influence of a small amount of methylfluorene, hydrogenation was performed at M/R adjusted to 0.035 mol % for 1^{st} run and at M/R adjusted to 0.14 mol % for subsequent runs, and the results thereof are shown in FIG. 21. Referring thereto, when using the catalyst for suppressing the formation of methylfluorene, hydrogenation was completed within about 40 minutes, and the hydrogen release efficiency of dehydrogenation also reached 97% after reaction for 4 hours, so the gravimetric H₂ storage density was maintained at 6.0 wt % or more in 10 experiments.

Hydrogen storage and hydrogen release tests were performed on para-monobenzyltoluene, selective isomer compositions (p72 and p55) obtained by a synthesis process of increasing the content of para-monobenzyltoluene, and MLH-sim and commercially available Marlotherm LH as comparative examples, and the results thereof are shown in FIG. 22 (hydrogenation; FIG. 22a; dehydrogenation; FIG. 22b). Referring thereto, the material that exhibited the best hydrogen storage/release efficiency was MBT-p72. Under the same reaction conditions, the t_{full} for hydrogenation was lowered to about 57% compared to when using commercially available Marlotherm LH, and dehydrogenation was also improved by about 19%.

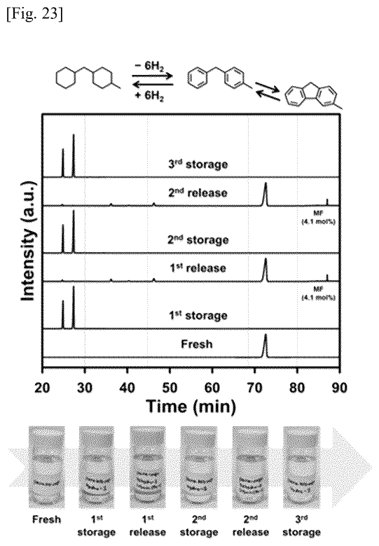

GC analysis of the products obtained by repeating hydrogen storage/release of para-monobenzyltoluene was performed, and the results thereof are shown in FIG. 23. Referring thereto, when using para-monobenzyltoluene, a quite large amount of methylfluorene was formed compared to when using the other isomers during dehydrogenation, so even though the amount of the catalyst was increased four times from the second hydrogenation, the hydrogen storage time increased from 44 minutes to 82 minutes. Therefore, rather than using para-monobenzyltoluene alone as the LOHC, the use of MBT-p72 with selectively para-isomer content is regarded as advantageous in view of lowering the manufacturing cost and saving the cost of the catalyst for hydrogenation due to the formation of methylfluorene.

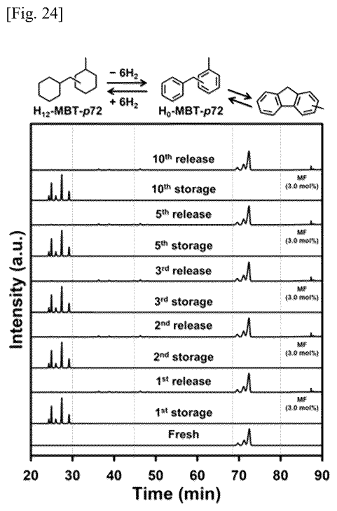

GC analysis of the products obtained by repeating hydrogen storage/release of the selective isomer composition (p72) prepared by a synthesis process of increasing the content of para-monobenzyltoluene was performed, and the results thereof are shown in FIG. 24. Referring thereto, when using the selective isomer composition (MBT-p72), the amount of methylfluorene formed during hydrogen release was 3.0 mol %, which is lower than when para-monobenzyltoluene was used alone (4.0 mol %). In particular, it is noteworthy that, during hydrogenation, the para-isomer took 82 minutes from the second hydrogenation under the same reaction conditions, but the time when using MBT-p72 was greatly shortened to 40 minutes, indicating excellent hydrogen storage/release efficiency.

Simple modifications or changes of the present disclosure may be easily used by those of ordinary skill in the art, and all such modifications or changes may be considered to be included in the scope of the present disclosure.

1. A process of reversibly storing and releasing hydrogen, comprising:
a) providing a monobenzyltoluene (MBT) isomer mixture having a para-isomer content of at least 50 wt %;
b) subjecting the monobenzyltoluene (MBT) isomer mixture to hydrogenation to store hydrogen; and
c) subjecting the hydrogenated monobenzyltoluene (MBT) isomer mixture to dehydrogenation to release hydrogen therefrom,
wherein hydrogen is stored or hydrogen is released by alternately performing hydrogenation and dehydrogenation, and
a methylfluorene content in the hydrogenated monobenzyltoluene (MBT) isomer mixture during dehydrogenation is maintained at less than 10 mol %.

2. The process of claim 1, wherein a halogen content in the monobenzyltoluene (MBT) isomer mixture is maintained at less than 30 ppm (w/w).

3. The process of claim 1, wherein the monobenzyltoluene (MBT) isomer mixture comprises 42 mol % or less of ortho-monobenzyltoluene (o-MBT), 15 mol % or less of meta-monobenzyltoluene (m-MBT), and 55 to 90 mol % of para-monobenzyltoluene (p-MBT).

4. The process of claim 1, wherein step b) is carried out in presence of a catalyst, and the catalyst comprises a support comprising a porous inorganic oxide, and at least one active metal selected from the group consisting of nickel, cobalt, palladium, platinum, iridium, ruthenium, rhodium, copper, gold, rhenium, zinc, molybdenum, tin, manganese, tungsten, and germanium.

5. The process of claim 4, wherein a content of the active metal in the catalyst used in step b) is determined in a range of 0.1 to 50 wt %.

6. The process of claim 4, wherein the porous inorganic oxide in the catalyst used in step b) is at least one selected from the group consisting of alumina, silica, silica-alumina, aluminum phosphate, zirconia, titania, ceria, and yttria.

7. The process of claim 1, wherein step c) is carried out in presence of a catalyst, and the catalyst comprises a support comprising a porous inorganic oxide, an active metal, and sulfur.

8. The process of claim 7, wherein, a content of the active metal in the catalyst used in step c) is determined in a range of 0.1 to 10 wt % and a sulfur content is determined in a range of 0.001 to 2 wt %.

9. The process of claim 8, wherein a molar ratio of sulfur/active metal in the catalyst used in step c) is determined in a range of 0.1 to 1.

10. The process of claim 7, wherein, in the catalyst used in step c), the porous inorganic oxide is alumina and the active metal is platinum.

11. The process of claim 1, wherein the monobenzyltoluene (MBT) isomer mixture exhibits a boiling point (1 atm) of 250 to 300° C. and a melting point of 10° C. or less.

12. The process of claim 11, wherein the monobenzyltoluene (MBT) isomer mixture exhibits a viscosity (20° C.) of 3 to 8 cP and a density (20° C.) of 0.94 to 1.02 g/cm³.

13. The process of claim 11, wherein a ratio (M/R) of active metal in a catalyst/LOHC during hydrogenation is adjusted in a range of 0.01 to 0.4 mol %, and a ratio of active metal in a catalyst/LOHC during dehydrogenation is adjusted in a range of 0.3 to 3 mol %.

14. The process of claim 1, wherein, during hydrogenation, a reaction temperature is determined in a range of 50 to 200° C. and a reaction pressure is determined in a range of 5 to 100 bar, and during dehydrogenation, a reaction temperature is determined in a range of 100 to 300° C. and a reaction pressure is determined in a range of 0.1 to 10 bar.

15. A liquid organic hydrogen carrier composition for reversible hydrogen storage/release, comprising:
42 mol % or less of ortho-monobenzyltoluene (o-MBT);
15 mol % or less of meta-monobenzyltoluene (m-MBT); and
55 to 90 mol % of para-monobenzyltoluene (p-MBT),
wherein a methylfluorene content is controlled to be less than 10 mol % and a halogen content is controlled to be less than 30 ppm (w/w).

16. The liquid organic hydrogen carrier composition of claim 15, wherein the halogen is chlorine.
